(19) United States
(12) Patent Application Publication
(10) Pub. No.: US 2016/0228015 A1
(43) Pub. Date: Aug. 11, 2016

(54) PHYSIOLOGICAL MONITORING DEVICE

(71) Applicants: Alireza Akhbardeh, Redwood City, CA (US); Amir Tehrani, San Francisco, CA (US); Ali Reza Zareh, Berkeley, CA (US)

(72) Inventors: Alireza Akhbardeh, Redwood City, CA (US); Amir Tehrani, San Francisco, CA (US); Ali Reza Zareh, Berkeley, CA (US)

(73) Assignee: Access Medical Sdn. Bhd., Kuala Lumpur (MY)

(21) Appl. No.: 14/615,438

(22) Filed: Feb. 6, 2015

(51) Int. Cl. A61B 5/0205; A61B 5/0452; A61B 5/00

(52) U.S. Cl. CPC A61B 5/02055; A61B 5/0006; A61B 5/0008; A61B 5/0452; A61B 5/7203; A61B 5/742; A61B 5/02405

(57) ABSTRACT

The present invention discloses a physiological monitoring device, comprising: a physiological signal acquisition block in the form of a capacitive touch panel for detecting user's physiological signals; a micro-processor block for capturing capacitance change in accordance with the user's physiological signal and estimating user's physiological data; and an application program for controlling the operation of physiological monitoring device.

### FIELD OF THE DISCLOSURE

The present invention generally relates to a physiological monitoring device, and methods for monitoring physiological data detectable through an electrical change using capacitive touch technologies commonly found in smart phones, tablets, computers, and other electronic devices.

### BACKGROUND OF THE DISCLOSURE

Certain physiological functions can be monitored through detection of electrical signals, e.g. electrocardiogram (ECG), impedance (used for detection of Congestive Heart Failure—CHF), and body fat.

These are currently being captured through a variety of specialized devices, such as Holter monitors for ECG measurement. Holter monitor is a portable device for continuous monitoring of ECG and heart rate. Each Holter system consists of two basic parts—a recorder for recording the signal and software for review and analysis of the record. The Holter system is connected to the patient's body through a number of electrodes. However, the main disadvantage of a Holter monitor is its application, often times being used after the patient has experienced adverse potential symptoms, at which time it is often a challenge to reproduce patient's symptoms in the short time the Holter monitor is worn. Furthermore, such devices are often cumbersome given the lead wires and patch electrodes frequently may disconnect during use.

Other specialized devices may use one or more leads or similar devices for capturing the electrical signal and communicating such signal to a mobile phone or other such devices for further analysis either on the mobile device or on a remote server.

Capacitive sensing as in capacitive touch screens are used in medical applications primarily as human interface devices in patient monitoring and as data display devices in medical applications. They have also been used in the past to track patient touch movement as indicators of muscle tremor. However, capacitive touchscreens have not been used to capture physiological signal with electrical changes, e.g. ECG, from human subjects.

Along with other types of specialized devices, the conventional medical monitoring devices may have several shortcomings:

1. The current systems require specialized devices, and are difficult to be integrated with every day tools such as smart phone or personal devices.

2. It is challenging for patients, specially the elderly, to properly place the leads and electrodes for such devices. Thus significant training is required for proper use.

3. Many physiological symptoms are short lived, and may no longer be detectable when the individual is connected to conventional monitoring devices post event.

It is therefore imperative to the extent possible to detect such symptoms using every day tools, such as smart phones, tablets and appliances and other equipment used on a routine basis and verily accessible without the need for additional leads, electrodes and other cumbersome devices.

### SUMMARY

Embodiments of the present disclosure provides for a mobile device and its working principle, to capture and monitor electrical signals from physiological conditions and either store and analyze the signals on the device or transmit the data via a communication network to a remote server for analysis.

In one embodiment, a physiological monitoring device comprises a capacitive touch sensor as a physiological signal acquisition block, the physiological signal is part of the touch signal; a micro-processor block for capturing and processing and estimating user's physiological signal from the captured touch data; an application program for controlling the operation of the device; and a data communication block to transmit the data via a communication network such as LAN, WAN & Wifi, mWifi, radio frequency (RF) or any other wireless medium to a remote location or to another mobile device such as a smart phone.

In another embodiment, the physiological signals are captured through a smart device, e.g. a smart phone. An application program may control the operations of the smart device for physiological signal capturing. In this case, the device may be activated to capture the touch signal for physiological signal analysis through any of various methods, including but not limited to specific touch buttons, touch patterns, or other key combinations. Once the device is activated and the touch signal captured, the captured touch signal shall then be processed to estimate the physiological data, either locally or remotely; and the data is stored and visualized on the smart device or remotely.

Further embodiments provides for common household devices using capacitive touch technologies, e.g. refrigerators, vacuum cleaners, etc., to store and analyze touch capacitance for physiological signal remotely or via a smart device.

### BRIEF DESCRIPTION OF THE DRAWINGS

FIG. 1 schematically illustrates the communication of the physiological monitoring device and the remote server embodying the principles of the present invention.

FIG. 2 schematically illustrates the workflow of the physiological monitoring device embodying the principles of the present invention.

FIG. 3 schematically illustrates the workflow of the micro-processor block of the physiological monitoring device embodying the principles of the present invention.

FIG. 4 schematically illustrates the structure of the physiological signal acquisition block embodying the principles of the present invention.

FIG. 5 schematically illustrates working principle of the micro-processor block embodying the principles of the present invention.

FIG. 6 schematically illustrates a credit card with the physiological monitoring device embodying the principles of the present invention.

### DETAILED DESCRIPTION

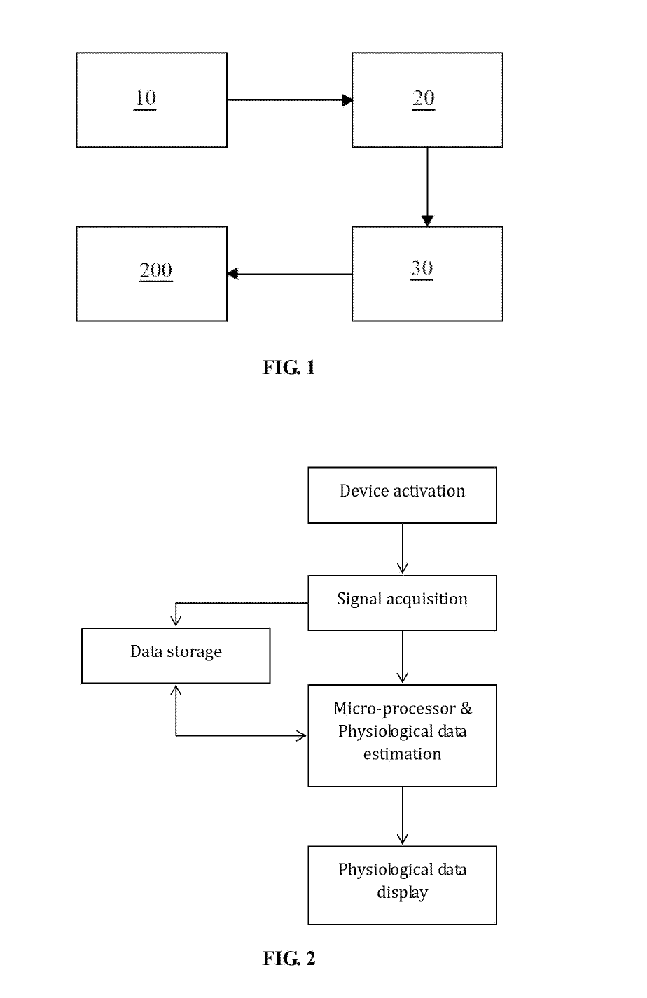

According to an embodiment of the present disclosure, a heart monitoring device is illustrated in accordance with FIG. 1.

Physiological monitoring device comprises physiological signal acquisition block 10, micro-processor (motherboard) block 20, and data communication block 30. The device further comprises an application program for operating the device, especially control of the various parameters for activating the device to initiate data capture, for estimating user's physiological data, communication to physicians or other relevant parties and display and storage of the data.

Physiological signal acquisition block 10 is a capacitive touch panel, commonly used in smart devices, smart phones, tablets and other devices which may include a protective or cosmetic cover layer. The capacitive touch panel 10 would also act as a human interface block. Generally, capacitive touch panel forms a capacitor consisting of two conductors, separated by an insulator. Two conductors may either be on different layers with a vertical distance between them or be on the same layer with a horizontal distance between them. In the case where two conductors are placed on the same layer, a capacitance between the two conductors are still formed and the change in the capacitance can be detected.

The capacitive touch panel of the present invention may utilize different type of capacitive touch technology, such as self capacitance technology and mutual capacitance. Take self capacitance technology for example, the capacitive touch panel is: electrode of a touch sensor in the touch panel represents one plate of such a capacitor. The second plate is represented by the environment of the sensor electrode forming a parasitic capacitor and another conductive object, such as a human finger for example to form touch capacitor. This capacitor is connected to a measurement circuit with the capacitance measured periodically. The measured capacitance increases when a conductive object approaches or touches the electrode. The change is detected by the measurement circuit and converted into a digital signal.

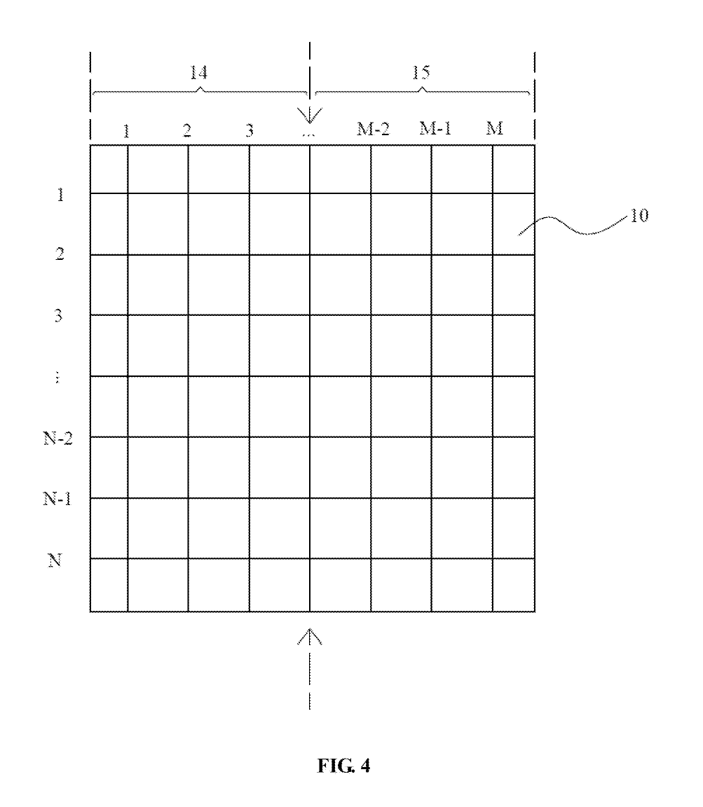

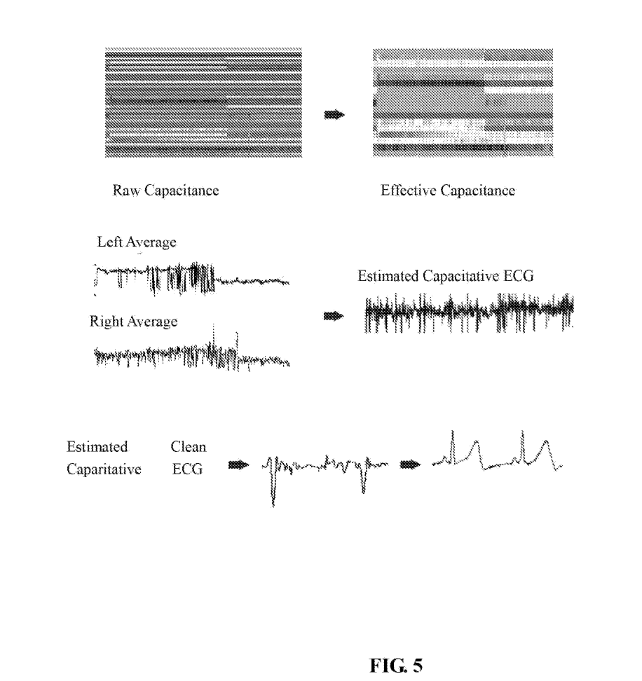

In the embodiment, as illustrated in FIG. 4, the touch panel is defined as a matrix with pluralities of rows and columns, for example, the number of column is M, and the number of row is N. The intersection of two adjacent rows and two adjacent columns respectively forms a cell.

A plurality of cells is formed on the matrix. The number of cells is the product of M and N. Physiological signal acquisition block 10 is capable of detecting user's touch, and touch movements, it comprises at least one capacitive touch sensor. The touch sensor is not only used for detecting touch coordinates generally, but also acts for detecting user's physiological signal as described previously. Particularly, the touch sensor is capable of detecting and capturing electrical changes in accordance with the user's touch. The touch signal includes the physiological signal, the user's ECG.

In another embodiment, mutual capacitance is used, the rows and columns of FIG. 4 used as driving and sensing lines. The driving lines carry current while the sensing lines detect changes in the electrical charge when the user places their finger(s) on the screen.

In some embodiments, the micro-processor (motherboard) block 20 is connected to physiological signal acquisition block 10. Micro-processor (motherboard) block 20 includes all necessary hardware (main and co-processors) and software and operating system to operate physiological signal acquisition block 10. It also has a signal processing utility function to process capacitance data from physiological signal acquisition block 10 and extracts touch location as well as capacitive physiological data (e.g. ECG) by use of signal processing methods that may include noise cancelation, filters, signal smoothing and amplification. Extracted physiological data is stored in the micro-processor block 20 (motherboard)'s flash memory 21 or remotely.

Data communication block 30 is capable of transmitting user's physiological data via a communication network such as LAN, WAN & Wifi, mWifi, radio frequency (RF) or any other wireless medium to remote server 200 such as a mobile device or other such devices, more specifically: a smart phone or computer tablet, and so on. Physiological signal acquisition block 10 may have a screen for displaying the estimated physiological data and the analysis result. Communication block 30 also may communicate with a hospital, a physician, or other care giver's computer or smartphone or other communication devices, relaying the raw physiological signal and/or analyzed physiological data. The communication with the hospital or other caregivers may be real-time or delayed depending on the severity of the condition.

In another embodiment, physiological signal acquisition block 10 may further be treated with medium for enhancing signal to noise ratios. Such medium may comprises gel (peelable or otherwise), saliva, water, Ag—Agcl or other medium that help electrical conductance as well as help improve signal quality.

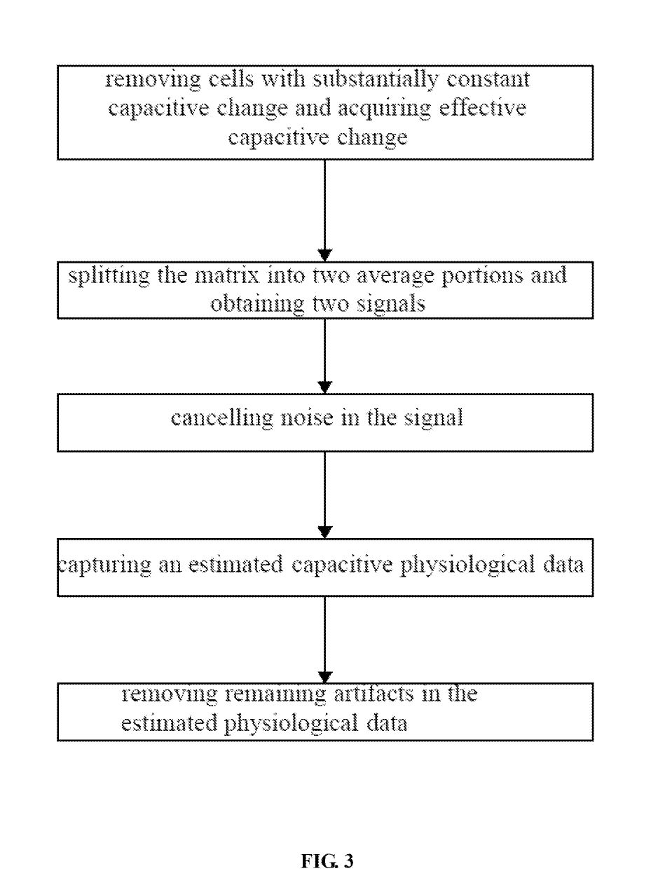

Please refer to FIG. 2, FIG. 3, FIG. 4, and FIG. 5, the working principle of physiological monitoring device is illustrated. FIG. 2 illustrates the overall work flow physiological monitoring device. FIG. 3 to FIG. 5 illustrate estimating a physiological data by micro-processor block 20. The application program for operating physiological monitoring device, the operating method comprises: firstly activates physiological monitoring device, when a user touches physiological monitoring device, electrical impulses are generated, the touch sensor of physiological signal acquisition block 10 detects user's electrical impulses, and acquires a touch signal, then physiological signal acquisition block 10 transmits the touch signal to micro-processor block 20. The touch signal indicates raw capacitance. After receiving the touch signal, micro-processor block 20 processes the signal to determine the physiological signal characteristics. The signal processing may include noise reduction, filtering and signal enhancement, as appreciated by anyone in the art. However the signal processing is not limited to these techniques only and as a person with ordinary skill in the art would appreciate, it may include other techniques to determine the physiological signal characteristics. In one embodiment, cells with substantially constant capacitance change are identified, then the cells with substantially constant capacitance are removed from matrix, that is neglecting the cells with substantially constant capacitance. Therefore the raw capacitance is transferred to an effective capacitance, and a value of effective capacitance change is obtained. The application program controls micro-processor block 20 splitting the matrix into two average portions, which is left portion 14 and right portion 15. Left portion is from column 1 to half of number M. Right portion is from half+1 to M. Therefore when a touch action occurs, two signals are obtained respectively from left portion 14 and right portion 15. Wherein signal of left portion is LeftAve(t))=Average(M(1:half)(t)), signal of right portion is RightAve(t)=Average(M(half+1:end(t)). The micro-processor block 20 applies a noise cancellation technique, such as recursive least square technique. The noise cancellation technique integrates the two signals as one signal, and cancels noises in the integrated signal. Certainly, in the present invention, other kinds of noise cancellation methods may be used to cancel the noise and arrive at the true physiological signal. So a physiological data is estimated. If there are remaining artifacts in the estimated physiological data filter 22 will remove the remaining artifacts to obtain an estimated clean physiological signal. After estimating the physiological data, micro-processor block 20 transmits the estimated physiological data to data communication block 30. Data communication block 30 transmits the physiological data to remote server 200.

Alternatively, after the physiological data is estimated, the application program further has a function of comparing the estimated physiological signal with a normal physiological pattern, e.g. analysis of ECG pattern to determine various potential heart conditions. The normal physiological patterns may be stored previously in micro-processor block 20's flash drive or remotely. Then the application program analyses the comparison to find out the estimated physiological data is normal or not, and indicate potential anomalies observed, e.g. based on ECG pattern and heart rhythm categorize if the patient has had an acute inferior myocardial infarction. Alternatively, physiological signal acquisition block 10 has a screen for displaying the estimated physiological data and the analysis result. So the results are displayed on the screen, or the physiological data are transmitted to remote server 200 for further analysis.

Alternatively, the user is not limited using his finger(s) to touch physiological monitoring device, other parts of his body can also touch physiological monitoring device for monitoring.

In another embodiment, a smart device such as a smart phone or a computer tablet with a capacitive touch screen may be configured to act as a physiological monitoring device. When the application program for physiological monitoring is not active, the touch panel acts as a normal human interface of smart device. As long as the application program is activated, the smart device will be the physiological signal acquisition block 10 and the micro-processor block 20 of physiological monitoring device capturing the touch signal and analyzing for physiological signal.

In another embodiment, a home appliance with a capacitive touch panel may be configured to act as a physiological monitoring device. The home appliance may be a vacuum cleaner, a refrigerator, and so on. The device will capture the touch signal upon activation, and send the captured signal to a smart device or remote server for analysis and display.

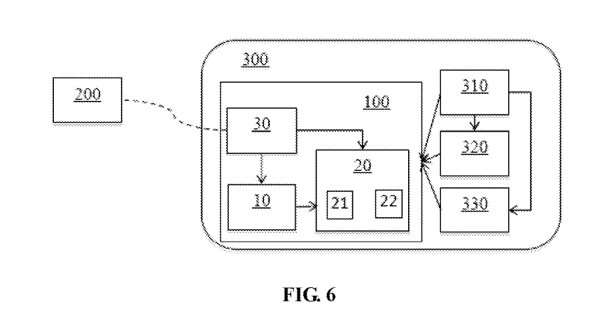

Please refer to FIG. 6, physiological monitoring device is capable of being integrated in a credit card form factor device 300. In this embodiment, credit card form factor device 300 has its own flat thin battery 310 usable for a defined number of activations or data storage time. Physiological signal acquisition block 10 is disposed on a fixed area on either sides of the credit card. The credit card further comprises reader 320, reader 320 is a bar code reader or a wireless data transferring reader. So that the estimated heart rate stored in the flash memory 21 of micro-processor block 20 can be retrieved by reader 320 and then transmitted to remote server 200. Activation mechanism 330 is integrated on the credit card form factor device 300, so when activation mechanism is activated, physiological monitoring device will be launched. Activation mechanism 330 may be a push button or a pressure sensor. Credit card form factor device 300 with physiological monitoring device will be disposable or reusable.

In another embodiment, it is capable of being integrated in a game controller. There is competition among video game console makers to further enhance the gaming experience. The integration of physiological data into gaming may allow gaming developers to enhance the gaming experience. In this embodiment, capacitive touch screen as physiological signal acquisition block is disposed on the game controllers, joy sticks or other video game controlling devices as another source of control input or to capture physiological signal. The physiological signal can be used in the game to adjust the intensity of the game or certain elements within the game to make the user's experience more exciting. The device may be activated to detect physiological signal including heart rate in response to the subject touching the touch screen or through gaming control software or other means.

When physiological monitoring device is used for game controller, it also has the function of communicating with remote server 200. The working principle is described in the previous embodiments.

The device, system, and method described in this patent application is capable of recording, capturing, detecting, displaying, and reporting ECG signals including one or more of the following parameters: heart rate, heart rate variability, P-wave, QRS, and T-wave.

The device, system, and method described in this patent application is also capable of recording, capturing, detecting, displaying, and reporting all physiological signals where an electrical current can be detected and measurable through human skin or touch including ECG, body impedance, and body temperature, and other possible signal captured through human skin interface.

Although the present disclosure has been disclosed above with reference to preferred embodiments thereof, it should be understood that the disclosure is presented by way of example only, and not limitation. Those skilled in the art can modify and vary the embodiments without departing from the spirit and scope of the present disclosure.

1. A physiological monitoring device, comprising:
a physiological signal acquisition block for detecting user's physiological signals;
a micro-processor block for capturing capacitance changes in accordance with the user's physiological signals and estimating user's physiological data;
an application program for controlling the operation of the device; and
a data communication block for transmitting data via wireless medium to a remote server or to another mobile device.

2. The physiological monitoring device according to claim 1, wherein the physiological signal acquisition block is a capacitive touch panel, comprising at least one capacitive sensor.

3. The physiological monitoring device according to claim 2, wherein the physiological signal acquisition block comprises a detachable medium for enhancing signal to noise ratios.

4. The physiological signal acquisition block according to claim 3, wherein the detachable medium comprises gel, saliva, water, and Ag—Agcl.

5. The physiological monitoring device according to claim 2, wherein the application program is capable of comparing an estimated physiological data with a normal physiological data and analyzing a comparison.

6. The physiological monitoring device according to claim 5, wherein the micro-processor block comprises a filter for removing remaining artifacts to obtain an estimated clean physiological signal.

7. The physiological monitoring device according to claim 6, wherein the micro-processor block further comprises a flash memory for storing the estimated physiological data.

8. The physiological monitoring device according to claim 7, wherein the micro-processor block is capable of processing the capacitive data from the physiological signal acquisition block and extracts a touch location as well as capacitive physiological data by use of noise cancelation and filters.

9. The physiological monitoring device according to claim 1, wherein the remote server is capable of communicating with the data communication block, and analyzing physiological data.

10. The physiological monitoring device according to claim 9, wherein the physiological signal acquisition block has a screen for displaying the estimated physiological data and the analysis result.

11. The physiological monitoring device according to claim 10, wherein the data communication block is capable of communicating with other communication devices, relaying a raw physiological signal or analyzed physiological data.

12. The physiological monitoring device according to claim 11, wherein it further comprises a detachable medium for enhancing signal to noise ratios.

13. The physiological monitoring device according to claim 11, wherein it is capable of being integrated in a credit card form factor device, and further comprises a reader for retrieving the estimated ECG signal including heart rate.

14. The physiological monitoring device according to claim 13, wherein the reader is a bar code reader or a wireless data transferring reader.

15. The physiological monitoring device according to claim 1, wherein it can be a diagnostic device.

16. The physiological monitoring device according to claim 11, wherein it can be a diagnostic device.

17. The physiological signals of claim 2, wherein the physiological signal is ECG including one or more of the following parameters: heart rate, heart rate variability, P-wave, QRS wave, T-wave; or the physiological data could be body impedance, body temperature, and other possible signals captured through human skin interface.

18. The physiological signals of claim 11, wherein the physiological signal is ECG including one or more of the following parameters: heart rate, heart rate variability, P-wave, QRS wave, T-wave; or the physiological data could be body impedance, body temperature, and other possible signals captured through human skin interface.

19. The physiological monitoring device according to claim 2, wherein it is capable of being integrated in a smart-device, or a house appliance.

20. The physiological monitoring device according to claim 11, wherein it is capable of being integrated in a smart-device or a house appliance.

21. The physiological monitoring device according to claim 11, wherein it is capable of being integrated in a game controller, for enhancing gaming experience.

22. A method of the application program, comprising:
detecting user's physiological signal and generating a touch signal;
transmitting the touch signal to a micro-processor block;
capturing a capacitance change and estimating the physiological data of user according to a capacitance change;
displaying a plurality of results on the screen or transmitting the physiological data to a remote server.

23. A method for operating a micro-processor block, wherein a capacitive touch screen is defined as a matrix with a plurality of rows and columns, and a plurality of cells are formed by an intersection of rows and columns, the method comprising:
analyzing cells with substantially constant capacitance change and obtaining effective capacitance change;
splitting the matrix into two average portions and obtaining two signals;
canceling noises in the signal;
capturing an estimated physiological data.

24. The method according to claim 23, wherein the noise is canceled by recursive least square technique.

25. The method according to claim 23, wherein it further comprises removing remaining artifacts in the estimated physiological data.

26. The physiological data captured in claim 23, wherein the physiological data could be ECG including one or more of the following parameters: heart rate, heart rate variability, P-wave, QRS wave, T-wave; or the physiological data could be body impedance, body temperature, and other possible signals captured through human skin interface.
